(19) United States
(12) Patent Application Publication
(10) Pub. No.: US 2016/0199280 A1
(43) Pub. Date: Jul. 14, 2016

(54) SURFACTANT COMPOSITION

(71) Applicants: KANEKA CORPORATION, Osaka-shi, Osaka (JP); NATIONAL INSTITUTE OF ADVANCED INDUSTRIAL SCIENCE AND TECHNOLOGY, Chiyoda-ku, Tokyo (JP)

(72) Inventors: Satohiro YANAGISAWA, Tokyo (JP); Masashi IZUMIDA, Takasago-shi (JP); Toshiaki TAIRA, Tsukuba-shi (JP); Tomohiro IMURA, Tsukuba-shi (JP); Dai KITAMOTO, Tsukuba-shi (JP)

(73) Assignees: Kaneka Corporation, Osaka-shi, Osaka (JP); National Institute of Advanced Industrial Science and Technology, Chiyoda-ku, Tokyo (JP)

(21) Appl. No.: 14/911,513

(22) PCT Filed: Aug. 11, 2014
(86) PCT No.: PCT/JP2014/071195

§ 371 Date: Feb. 11, 2016

(30) Foreign Application Priority Data
Aug. 12, 2013 (JP) 2013-167765

(51) Int. Cl. A61K 8/64; A61Q 5/00; A61K 8/02; A61K 8/41; A61K 8/14

(52) U.S. Cl. CPC A61K 8/64; A61K 8/416; A61K 8/14; A61K 8/0291; A61Q 5/00; A61K 2800/70; A61K 2800/596

(57) ABSTRACT

An objective of the present invention is to provide a surfactant composition which hardly causes the formation of an insoluble salt and which contains a safe and extremely stable micelle or vesicle. Also, an objective of the present invention is to provide a hair cosmetic material which contains the surfactant composition. The surfactant composition according to the present invention is characterized in comprising a micelle or a vesicle which contain surfactin and a specific cationic surfactant.

### TECHNICAL FIELD

The present invention relates to a surfactant composition which contains a micelle or a vesicle which is formed from an anionic surfactant and a cationic surfactant, and a hair cosmetic material which contains the surfactant composition.

### BACKGROUND ART

It is known that a cationic surfactant such as a quaternary ammonium salt and a tertiary ammonium salt has an effect of protecting hair by being adsorbed to a damaged portion of hair. On the one hand, a cationic surfactant has a problem of having significant skin irritancy.

In contrast, when an anionic surfactant and a cationic surfactant are used in combination, less skin irritation is caused as compared with the case of using a cationic surfactant only. There is therefore a possibility that a damaged portion of hair may be protected while suppressing skin irritation in such a case.

However, in particular, when an anionic surfactant and a cationic surfactant are used in combination in equimolar amounts or approximately equimolar amounts, both of the surfactants are strongly bonded to one another by electrostatic attractive force, leading to a problem of the formation and precipitation of an insoluble salt.

In the inventions of Patent Document 1 and Non-Patent Document 1, a specific quaternary ammonium salt is used as a cationic surfactant to achieve a solution for such a problem. In addition, in the invention of Patent Document 2, a vesicle which contains a ceramide and a cationic surfactant having two long-chain alkyl groups is utilized for a hair cosmetic material. A technique for forming a vesicle of a pseudo double chain-type surfactant by using a combination of an anionic surfactant and a cationic surfactant has been known before the prior arts.

### PRIOR ART DOCUMENT

### Patent Document

Patent Document 1: JP 2011-131137 A

Patent Document 2: JP 2007-191410 A

Non-patent Document 1: ERIC W. KALER et al., SCIENCE, Vol. 245, pp. 1371-1374 (1989)

### DISCLOSURE OF THE INVENTION

### Problems to be Solved by the Invention

As described above, it has been known to use a surfactant composition which contains a vesicle for a hair cosmetic material. Patent Document 1 discloses also a problem that a cationic surfactant and an anionic surfactant are strongly bonded to form an insoluble salt.

However, when a conventional cationic surfactant and an anionic surfactant, of which hydrophilic group parts are small, are combined each other, a problem still exists that the cation and anion in the hydrophilic group parts are strongly bonded by electrostatic attractive force and hence an insoluble salt is formed. In addition, it is necessary to also suppress harm due to skin irritation or the like caused by a common surfactant.

Under such circumstances, an objective of the present invention is to provide a surfactant composition which hardly causes the formation of an insoluble salt and which contains a safe and extremely stable micelle or vesicle. Also, an objective of the present invention is to provide a hair cosmetic material which contains the surfactant composition.

### Means for Solving the Problems

The inventors of the present invention made extensive studies to solve the above problems. As a result, the inventors completed the present invention by finding that an extremely stable micelle or vesicle is formed when surfactin, which is a natural surfactant, and a specific cationic surfactant are used in combination.

Hereinafter, the present invention is described.

[1] A surfactant composition,

comprising a micelle or a vesicle,

wherein the micelle or vesicle contains surfactin represented by the following formula (I):

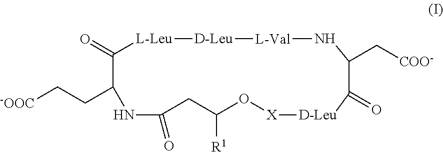

wherein ‘X’ is a residue of an amino acid selected from leucine, isoleucine and valine; and R¹ is a C₉₋₁₈ alkyl group, and a cationic surfactant having two C₈₋₂₀ alkyl groups or a cationic surfactant having one C₈₋₂₀ alkyl group.

[2] The surfactant composition according to the above [1], wherein the cationic surfactant having two C₈₋₂₀ alkyl groups is represented by the following formula (II):

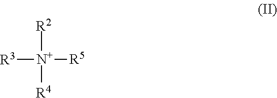

wherein R² and R³ are independently C₈₋₂₀ alkyl groups; R⁴ and R⁵ are independently C₁₋₄ alkyl groups; and R⁴ and R⁵ may bind to one another to form a ring.

[3] The surfactant composition according to the above [1] or [2], wherein the cationic surfactant having one C₈₋₂₀ alkyl group is represented by the following formula (III) or (IV):

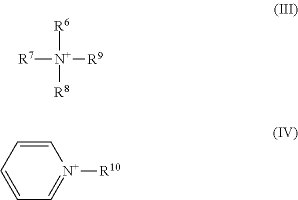

wherein R⁶ is a C₈₋₂₀ alkyl group; R⁷ and R⁹ are independently C₁₋₄ alkyl groups; R¹⁰ is a C₈₋₂₀ alkyl group; and two of R⁷ to R⁹ may bind to one another to form a ring.

[4] The surfactant composition according to the above [1], comprising the vesicle containing the surfactin (I) and the cationic surfactant having two C₈₋₂₀ alkyl groups. The cationic surfactant having two C₈₋₂₀ alkyl groups and the surfactin, which has one long-chain alkyl group, form a pseudo triple chain-type complex. Such a complex has not been known and may exhibit a behavior and physical properties which are different from those of a conventional pseudo double chain-type complex. In addition, a very stable vesicle may readily formed.

[5] The surfactant composition according to any one of the above [1] to [4], wherein the molar ratio of the cationic surfactant having two C₈₋₂₀ alkyl groups or cationic surfactant having one C₈₋₂₀ alkyl group to the surfactin (I) is not less than 0.1 and not more than 10. When the molar ratio is included in the above range, a micelle or vesicle can be formed more certainly. In addition, there is generally a problem that an insoluble salt is readily formed to be precipitated when an anionic surfactant and a cationic surfactant are used in combination in equimolar amounts or approximately equimolar amounts. However, in the case of the combination according to the present invention, even when such surfactants are used in equimolar amounts or approximately equimolar amounts, a very stable micelle or vesicle is formed and such an insoluble salt is hardly produced due to the unique bulky cyclic peptide structure of the surfactin (I). In addition, when the above-described molar ratio is included in the above range, skin irritation by a cationic surfactant can be inhibited and additionally, the effect to protect a damaged part of hair can be sufficiently obtained.

[6] The surfactant composition according to any one of the above [1] to [5], further comprising an aqueous solvent, and wherein the micelle or vesicle containing the surfactin (I) and the cationic surfactant having two C₈₋₂₀ alkyl groups or cationic surfactant having one C₈₋₂₀ alkyl group is dispersed in the aqueous solvent. The composition is extremely stable, since precipitate is hardly produced; therefore, the composition is very excellent as a product.

[7] A hair cosmetic material, comprising the surfactant composition according to any one of the above [1] to [6].

### Effect of the Invention

Since the surfactin (I), which is one component of the surfactant composition according to the present invention, has a bulky cyclic peptide portion, an insoluble salt is hardly formed and hence a micelle and a vesicle having a regular structure tend to be formed easily due to moderate electrostatic interaction of the surfactin (I) with a cationic surfactant. In addition, an alkyl chain tends to have a proper packing structure easily in such a micelle and vesicle. Thus, the micelle or vesicle which is contained in the surfactant composition according to the present invention is extremely stable, and a product which contains the surfactant composition according to the present invention has high stability. Furthermore, since the surfactin (I), which is one component of the surfactant composition according to the present invention, is safe due to low skin irritancy, the surfactant composition according to the present invention is also safe with low skin irritancy. For example, the surfactant composition according to the present invention is therefore useful as a safe hair cosmetic material having lower skin irritancy, since the cationic surfactant which is one component of the surfactant composition according to the present invention has not only a hair protective action as described above but also excellent in antibacterial activity.

In addition, the stable micelle and vesicle can respectively contain a hydrophobic or hydrophilic active ingredient or the like. The surfactant composition according to the present invention therefore can be used not only for a hair cosmetic material, a germicidal disinfectant or a disinfectant detergent but also for a drug delivery system, and is extremely useful for industries.

### BRIEF DESCRIPTION OF THE DRAWINGS

FIG. 1 are a differential interference microscope image and a polarizing microscope image of a vesicle which is composed of a surfactin/didecyldimethylammonium chloride composition.

FIG. 2 is a particle size distribution of a micelle which is composed of a surfactin/decyltrimethylammonium chloride composition.

### MODE FOR CARRYING OUT THE INVENTION

The surfactant composition according to the present invention is characterized in comprising the micelle or vesicle which contains the surfactin (I) and the specific cationic surfactant.

The surfactant composition according to the present invention essentially contains surfactin represented by the following formula (I). The surfactin (I) has a small environmental load and safety to a human body, since the surfactin (I) is a peptide compound.

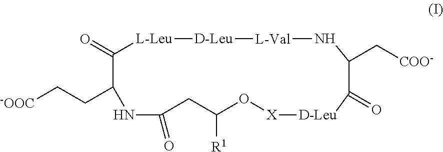

wherein ‘X’ is a residue of an amino acid selected from leucine, isoleucine and valine; and R¹ is a C₉₋₁₈ alkyl group.

Although the amino acid residue as ‘X’ may be either in a L-form or a D-form, the L-form is preferred.

The term “C₉₋₁₈ alkyl group” means a linear or branched monovalent saturated hydrocarbon group having not less than 9 and not more than 18 carbon atoms. The example thereof includes n-nonyl, 6-methyloctyl, 7-methyloctyl, n-decyl, 8-methylnonyl, n-undecyl, 9-methyldecyl, n-dodecyl, 10-methylundecyl, n-tridecyl, 11-methyldodecyl, n-tetradecyl, n-pentadecyl, n-hexadecyl, n-heptadecyl and n-octadecyl.

Either one of the surfactin (I) may be used or not less than two of the surfactin (I) may be used. For example, two or more surfactin (I) of which C₉₋₁₈ alkyl groups as R¹ are different may be used.

The surfactin (I) can be isolated from a culture broth prepared by culturing a microorganism such as a strain belonging to Bacillus subtilis in accordance with a known method. The surfactin (I) may be a purified product or an unpurified product. For example, a culture broth may be directly used as the unpurified product. Alternatively, the product of the surfactin (I) obtained by a chemical synthesis method may be similarly used.

The cationic surfactant which is an essential component of the surfactant composition according to the present invention has two C₈₋₂₀ alkyl groups or one C₈₋₂₀ alkyl group.

The term “C₈₋₂₀ alkyl group” means a linear or branched monovalent saturated hydrocarbon group having not less than 8 and not more than 20 carbon atoms. The example thereof includes n-octyl, isooctyl, 2-ethylhexyl, n-nonadecyl, isononadecyl, n-icosyl and isoicosyl in addition to the above-described C₉₋₁₈ alkyl group.

The C₈₋₂₀ alkyl group constitutes the hydrophobic portion of the cationic surfactant. When a cationic surfactant having two C₈₋₂₀ alkyl groups is used with the surfactin (I) in combination, the cationic group of the cationic surfactant and the anionic group of the surfactin (I) are bonded to one another by electrostatic attractive force to form a pseudo triple chain-type structure. The packing becomes dense and a strong and stable vesicle is formed due to such a pseudo triple chain-type structure in comparison with a general pseudo double chain-type structure. The vesicle coats hair surface in higher density due to the pseudo triple chain-type structure; as a result, tangle of hairs caused by electrostatic attractive force may be remarkably reduced. In addition, the vesicle may incorporate a water-soluble active ingredient or the like inside in an aqueous solvent dispersion.

On the one hand, when a cationic surfactant having one C₈₋₂₀ alkyl group is used with the surfactin (I) in combination, a pseudo double chain-type structure is obtained to form a micelle. A dispersion of general pseudo double chain-type is turbid; however, the micelle dispersion according to the present invention is transparent by visual observation. Although the reason is unknown, it is considered that very fine micelles are formed. The micelle may incorporate a hydrophobic active ingredient or the like inside in an aqueous solvent dispersion. In addition, the micelle is very useful as a formulation ingredient, since the micelle dispersion is transparent.

The cationic surfactant having two C₈₋₂₀ alkyl groups is exemplified by the compound represented by the following formula (II):

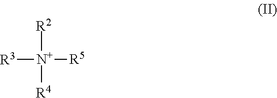

wherein R² and R³ are independently C₈₋₂₀ alkyl groups; R⁴ and R⁵ are independently C₁₋₄ alkyl groups; and R⁴ and R⁵ may bind to one another to form a ring.

The cationic surfactant having one C₈₋₂₀ alkyl group is exemplified by the compound represented by the following formula (III) or (IV):

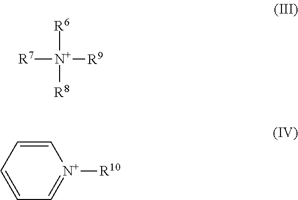

wherein R⁶ is a C₈₋₂₀ alkyl group; R⁷ and R⁹ are independently C₁₋₄ alkyl groups; R¹⁰ is a C₈₋₂₀ alkyl group; and two of R⁷ to R⁹ may bind to one another to form a ring.

The term “C₁₋₄ alkyl group” means a linear or branched monovalent saturated hydrocarbon group having not less than 1 and not more than 4 carbon atoms. The example thereof includes methyl, ethyl, n-propyl, isopropyl, n-butyl, isobutyl, sec-butyl and t-butyl.

When the R⁴ and R⁵ or two of R⁷ to R⁹ bind to one another to form a ring, the ring is exemplified by a piperidine ring and a pyrrolidine ring.

As the cationic surfactant, a quaternary ammonium germicidal disinfectant such as benzalkonium chloride and benzethonium chloride may be used except for the above-described cationic surfactant.

Either one of the above-described cationic surfactants may be used or not less than two of the surfactants may be used. In addition, the cationic surfactant having two C₈₋₂₀ alkyl groups and the cationic surfactant having one C₈₋₂₀ alkyl group may be used in combination. In other words, the term “a cationic surfactant having two C₈₋₂₀ alkyl groups or a cationic surfactant having one C₈₋₂₀ alkyl group” means one or more cationic surfactants selected from a cationic surfactant having two C₈₋₂₀ alkyl groups and a cationic surfactant having one C₈₋₂₀ alkyl group.

An amount of the surfactin (I) and the above-described cationic surfactant to be used may be appropriately adjusted in the range where a micelle or a vesicle is formed. For example, the molar ratio of the cationic surfactant having two C₈₋₂₀ alkyl groups or cationic surfactant having one C₈₋₂₀ alkyl group to the surfactin (I) may be adjusted to not less than 0.1 and not more than 10. The molar ratio is more preferably not less than 0.2, even more preferably not less than 0.5, and more preferably not more than 8, even more preferably not more than 5, particularly preferably not more than 2.

Amounts of the surfactin (I) and the above-described cationic surfactant to be used are preferably equimolar or approximately equimolar each other. For example, when the above-described cationic surfactant in an excessive amount to the surfactin (I) is applied to a human body, skin irritation may be possibly caused. In addition, for example, when the surfactin (I) in an excessive amount to the above-described cationic surfactant is applied to hair, protective effect may not be possibly exerted to a satisfactory extent. On the one hand, when the above surfactants are used in equimolar amounts or approximately equimolar amounts, a micelle or vesicle is successfully formed and an effect by a cationic surfactant may be sufficiently exerted with inhibiting the above-described skin irritation. Specifically, it is particularly preferred that a molar ratio of the above-described cationic surfactant to the surfactin (I) is adjusted to not less than 0.8 and not more than 1.2.

The above-described surfactin (I) and cationic surfactant are bonded each other in an aqueous solvent to form a micelle or vesicle due to electrostatic attractive force.

The aqueous solvent is exemplified by water and a mixed solvent of water and a water-miscible solvent. The water-miscible solvent to be used in the present invention is exemplified by an alcohol solvent such as methanol, ethanol and isopropanol; an ether solvent such as tetrahydrofuran; an amide solvent such as dimethylformamide and dimethylacetamide; and a sulfoxide solvent such as dimethylsulfoxide. Since a water-miscible solvent may possibly inhibit the formation of a micelle and vesicle or cause skin irritation, a ratio of a water-miscible solvent to an aqueous mixed solvent is preferably not more than 50 mass %, more preferably not more than 25 mass %, even more preferably not more than 10 mass %, and particularly preferably not more than 5 mass %. The lower limit of the ratio is not particularly restricted, and may be not less than 0.5 mass %, preferably not less than 1 mass %, and more preferably not less than 2 mass %.

When a higher alcohol of which carbon number is not less than 6 and not more than 20 is added, the formation of a micelle or vesicle may be accelerated.

The surfactant composition according to the present invention can be produced without difficulty by mixing the surfactin (I) and the above-described cationic surfactant in an aqueous solvent. For example, the above-described cationic surfactant is mixed in an aqueous solution of the surfactin (I).

As the surfactin (I), a salt thereof may be used. Specifically, a salt of the surfactin (I) at least one of which two carboxy groups is electrostatically bound to an alkali metal ion or an ammonium ion can be used. Such a salt is very convenient due to a superior solubility in an aqueous solvent.

An alkali metal ion to be used for the salt of the surfactin (I) is not particularly restricted, and is exemplified by a lithium ion, a sodium ion, a potassium ion and the like. When two or more alkali metal ions are used, the ions are the same or different from each other.

A substituent of an ammonium ion is exemplified by a C₁₋₄ alkyl group such as methyl, ethyl, n-propyl, isopropyl, n-butyl and t-butyl; a C₇₋₁₄ aralkyl group such as benzyl, methylbenzyl and phenylethyl; and a C₆₋₁₂ aryl group such as phenyl, toluyl and xylyl. An ammonium ion is exemplified by a tetramethylammonium ion, a tetraethylammonium ion, a pyridinium ion and the like.

Two counter cations in the salt of the surfactin (I) may be the same or different from each other. In addition, one of the carboxy groups may be in the state of —COOH or —COO⁻.

The above-described cationic surfactant to be used may be in the condition of a salt. The counter anion thereof is exemplified by a halide ion such as a chloride ion, a bromide ion and an iodide ion; an inorganic acid ion such as a nitrate ion, a phosphate ion, a sulfate ion and a hydrogensulfate ion; and an organic acid ion such as an acetate ion, a trifluoroacetate ion, a methanesulfonate ion, a toluenesulfonate ion and a trifluoromethanesulfonate ion.

The concentration of the aqueous solvent solution of the salt of the surfactin (I) maybe appropriately adjusted, and for example, may be adjusted to not less than about 1 mM and not more than 50 mM or not less than about 0.1 mass % and not more than about 5 mass %.

Then, the above-described cationic surfactant in an appropriate amount is added to be mixed in the aqueous solvent solution of the salt of the surfactin (I). The condition at the time may be appropriately selected, and for example, the temperature maybe an ordinary temperature. Specifically, the temperature may be adjusted to not less than about 10° C. and not more than about 50° C. In addition, in the present invention, the mixture may be moderately stirred, since a micelle or vesicle is easily formed without adding an external force such as ultrasonic waves. The stirring time may be adjusted to not less than about 10 minutes and not more than about 5 hours. In general, a micelle and vesicle are formed by adding an external force such as ultrasonic waves. However, in such a case, it is very difficult to produce a micelle and vesicle which have even particle size and which is excellent in dispersion stability with high reproducibility. On the one hand, the vesicle and micelle according to the present invention is thermodynamically stable and excellent in dispersion stability, and the particle diameter thereof is narrow, since the vesicle and micelle can be produced by moderate stirring treatment without an external force. In addition, a vesicle and micelle which have the same characteristics can be produced at high reproducibility.

The surfactin composition according to the present invention can be applied to various products which are exemplified by a hair cosmetic material such as a hair conditioner, a hair treatment product, a hair pack, a hair cream and a hair lotion; a sterilizing disinfectant; and a sterilizing detergent. In such a product, the dispersion of the obtained above-described micelle or vesicle may be concentrated, and other component may be added depending on the use application.

The other component is not particularly restricted, and is exemplified by a polyol such as ethylene glycol, propylene glycol, glycerin and sorbitol; a polysaccharide thickener such as guar gum and xanthane gum; a cellulose compound such as hydroxypropyl cellulose and carboxymethyl cellulose; a carboxyvinyl polymer such as an acrylic acid polymer and an acrylic acid copolymer; a silicone compound; a non-ionic surfactant solution; a colorant; a pH adjuster; a plant extract; a preservative; a chelating agent; a vitamin preparation; a medicinal ingredient such as an anti-inflammatory drug; a fragrance; a ultraviolet absorber; an antioxidant; a sphingolipid such as ceramide; an oil such as olive oil, coconut oil, palm oil and canola oil.

The present application claims the benefit of the priority dates of Japanese patent application No. 2013-167765 filed on Aug. 12, 2013. All of the contents of the Japanese patent application No. 2013-167765 filed on Aug. 12, 2013, are incorporated by reference herein.

### EXAMPLES

Hereinafter, the present invention is described in more detail with Examples. However, the present invention is not restricted to the following Examples in any way, and it is possible to work the present invention according to the Examples with an additional appropriate change within the range of the above descriptions and the following descriptions. Such a changed embodiment is also included in the technical scope of the present invention.

### Example 1

### Preparation of Pseudo Triple Chain-Type Surfactant Vesicle

Surfactin sodium salt having C₉₋₁₈ long-chain alkyl groups was dissolved in ultrapure water to produce 19 mM solution (5 mL). The concentration corresponds to about 2 mass %. Hereinafter, surfactin sodium salt is referred to as “SFNa”. Into the solution, a cationic surfactant having two long-chain alkyl groups was added in an equimolar. The mixture was moderately stirred at room temperature for 1 hour without adding an external force such as ultrasonic waves. Then, the reaction mixture was observed. As the cationic surfactant having two long-chain alkyl groups, dimethyldidodecylammonium chloride, dimethyldidecylammonium chloride and dimethyldioctylammonium chloride were used. The result is shown in Table 1.

TABLE 1

| Composition | | | | |
|---|---|---|---|---|
| No. | Composition | Structure | Condition | Viscosity |
| 1 | SFNa/dimethyldidodecylammonium chloride | pseudo triple | white/ | low |
| | | chain-type | turbid | |
| 2 | SFNa/dimethyldidecylammonium chloride | pseudo triple | white/ | low |
| | | chain-type | turbid | |
| 3 | SFNa/dimethyldioctylammonium chloride | pseudo triple | white/ | low |
| | | chain-type | turbid | |

### Test Example 1

### Stability Test

The dispersions of the pseudo triple chain-type surfactant vesicle obtained in Example 1 were turbid in white. The dispersions were left to stand at room temperature for 4 months. As a result, it was demonstrated that molecular aggregates which have high stability were formed in all of the dispersions, since the formation of precipitate was not observed.

### Test Example 2

### Observation of Condition

As the above-described Example 1, SFNa was dissolved in ultrapure water to produce 4.8 mM solution (5 mL). The concentration corresponds to about 0.5 mass %. Into the solution, dimethyldidecylammonium chloride was added in an equimolar. The mixture was moderately stirred at room temperature for 1 hour to produce a white dispersion.

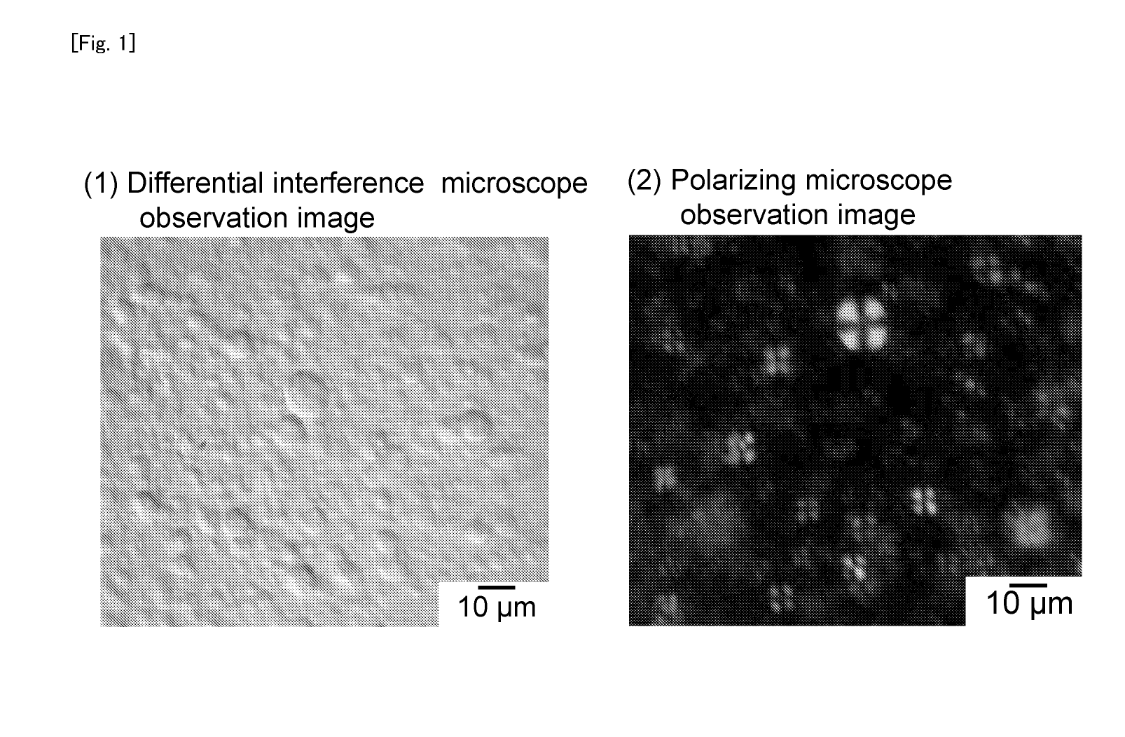

The dispersion was thinly spread on a preparation to be observed by a polarizing microscope (product name: ECLIPSE E600, manufactured by Nikon Corporation) equipped with a CCD camera (product name: DS-5M, manufactured by Nikon Corporation). Also, differential interference observation was carried out using the microscope. The result of differential interference microscope observation is shown in FIG. 1(1), and the result of polarizing microscope observation is shown in FIG. 1(2).

As FIG. 1, when the sample was observed using a differential interference microscope, particles having diameters of several micrometers to dozens of micrometers were confirmed. When the same sample was observed using a polarizing microscope, Maltese cross was observed under crossed Nicols condition. Thus, it was demonstrated that a vesicle was formed.

### Example 2

### Preparation of Pseudo Double Chain-Type Surfactant Micelle

A SFNa/cationic surfactant composition was prepared similarly to the above-described Example 1 except that a cationic surfactant having one long-chain alkyl group was used in place of a cationic surfactant having two long-chain alkyl groups. As the cationic surfactant having one long-chain alkyl group, dodecyltrimethylammonium chloride, decyltrimethylammonium chloride and octyltrimethylammonium chloride were used. The observation result of the characteristics is shown in Table 2.

TABLE 2

| Composition | | | | |
|---|---|---|---|---|
| No. | Composition | Structure | Condition | Viscosity |
| 4 | SFNa/dodecyltrimethylammonium chloride | pseudo double | colorless/ | high |
| | | chain-type | transparent | |
| 5 | SFNa/decyltrimethylammonium chloride | pseudo double | colorless/ | low |
| | | chain-type | transparent | |
| 6 | SFNa/octyltrimethylammonium chloride | pseudo double | colorless/ | low |
| | | chain-type | transparent | |

In particular, in the case of the combination of SFNa/dodecyltrimethylammonium chloride, it was suggested that a string-like micelle was formed since the obtained solution was transparent and highly viscous.

### Test Example 3

### Stability Test

The dispersions of the pseudo double chain-type surfactant micelle obtained in Example 2 were colorless and transparent. The dispersions were left to stand at room temperature for 3 months. As a result, it was demonstrated that molecular aggregates which have high stability were formed in all of the dispersions, since the formation of precipitate was not observed.

### Test Example 4

### Confirmation of Micelle Formation

As the above-described Example 1, SFNa was dissolved in ultrapure water to produce 19 mM solution (5 mL). The concentration corresponds to about 2 mass %. Into the solution, decyltrimethylammonium chloride was added in an equimolar. The mixture was moderately stirred at room temperature for 1 hour.

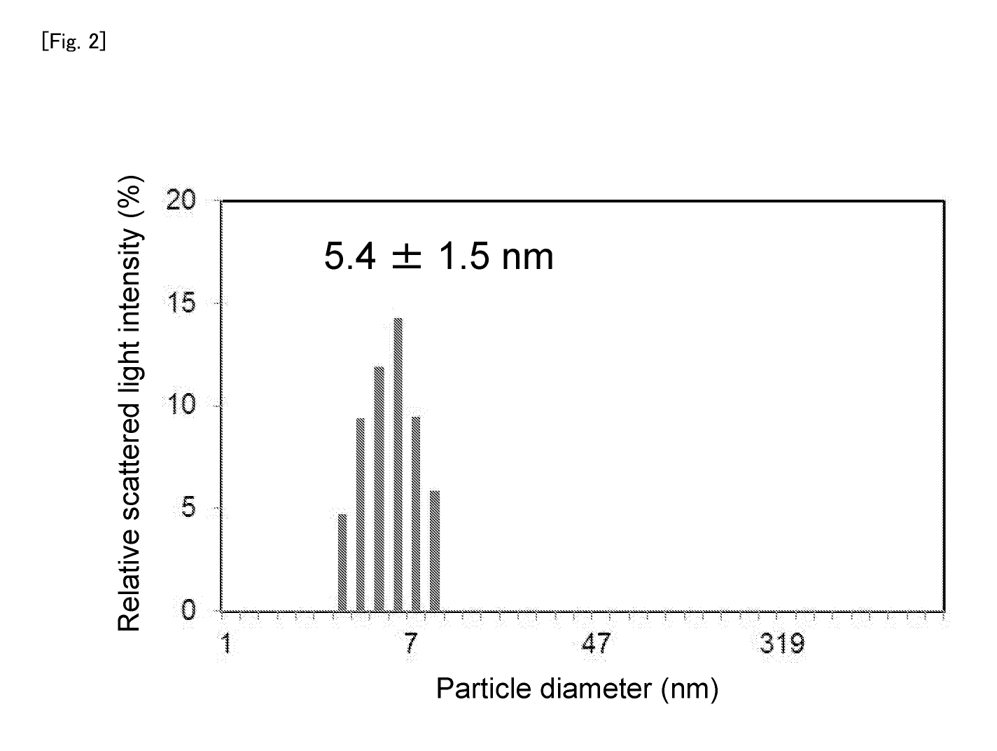

The obtained transparent dispersion (5 mL) was added into a glass cell, and the size distribution of the particles which were contained in the dispersion was measured by a dynamic light scattering measuring device (product name: DLS-7000, manufactured by OTSUKA ELECTRONICS Co., LTD.). Ar laser of which λ was 488 nm was used as a light source, and the scattering angle was adjusted to 90°. The result is shown in FIG. 2.

As FIG. 2, it was demonstrated that micelles of which average particle diameter was 5.4±1.5 nm was formed.

1. A surfactant composition,
comprising a micelle or a vesicle,
wherein the micelle or vesicle contains surfactin represented by the following formula (I): 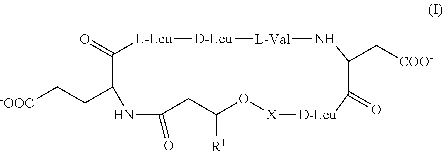
wherein ‘X’ is a residue of an amino acid selected from leucine, isoleucine and valine; and R¹ is a C₉₋₁₈ alkyl group,
and a cationic surfactant having two C₈₋₂₀ alkyl groups or a cationic surfactant having one C₈₋₂₀ alkyl group.

2. The surfactant composition according to claim 1, wherein the cationic surfactant having two C₈₋₂₀ alkyl groups is represented by the following formula (II): 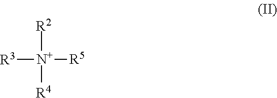
wherein R² and R³ are independently C₈₋₂₀ alkyl groups; R⁴ and R⁵ are independently C₁₋₄ alkyl groups; and R⁴ and R⁵ may bind to one another to form a ring.

3. The surfactant composition according to claim 1, wherein the cationic surfactant having one C₈₋₂₀ alkyl group is represented by the following formula (III) or (IV): 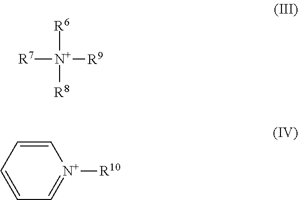
wherein R⁶ is a C₈₋₂₀ alkyl group; R⁷ and R⁹ are independently C₁₋₄ alkyl groups; R¹⁰ is a C₈₋₂₀ alkyl group; and two of R⁷ to R⁹ may bind to one another to form a ring.

4. The surfactant composition according to claim 1, comprising the vesicle containing the surfactin (I) and the cationic surfactant having two C₈₋₂₀ alkyl groups.

5. The surfactant composition according to claim 1, wherein the molar ratio of the cationic surfactant having two C₈₋₂₀ alkyl groups or cationic surfactant having one C₈₋₂₀ alkyl group to the surfactin (I) is not less than 0.1 and not more than 10.

6. The surfactant composition according to claim 1, further comprising an aqueous solvent, and wherein the micelle or vesicle containing the surfactin (I) and the cationic surfactant having two C₈₋₂₀ alkyl groups or cationic surfactant having one C₈₋₂₀ alkyl group is dispersed in the aqueous solvent.

7. A hair cosmetic material, comprising the surfactant composition according to claim 1.

8. A hair cosmetic material, comprising the surfactant composition according to claim 4.

9. A hair cosmetic material, comprising the surfactant composition according to claim 6.
